(19) United States
(12) Patent Application Publication
(10) Pub. No.: US 2010/0293251 A1
(43) Pub. Date: Nov. 18, 2010

(54) Asynchronous Method for Obtaining Spectacle Features to Order

(75) Inventors: Saurphéa Suy, Charenton-Le-Pont (FR); Philippe Clara, Charenton-Le-Pont (FR); Sébastien Chailley, Charenton-Le-Pont (FR)

Correspondence Address: COHEN, PONTANI, LIEBERMAN & PAVANE LLP, 551 FIFTH AVENUE, SUITE 1210, NEW YORK, NY, 10176, US

(73) Assignee: ESSILOR INTERNATIONAL (COMPAGNIE GENERALE D'OPTIQUE), Charenton Le Pont (FR)

(21) Appl. No.: 12/811,029

(22) PCT Filed: Dec. 23, 2008
(86) PCT No.: PCT/EP08/68273

§ 371 Date: Jun. 28, 2010

(30) Foreign Application Priority Data
Dec. 28, 2007 (EP) 07301754.3

(51) Int. Cl. G06F 17/30; G06F 15/16; A61B 3/10

(52) U.S. Cl. 709/219; 707/803; 351/204; 707/E17.044

(57) ABSTRACT

A method and a computer system and its components for providing a spectacle frame adapted to a future wearer. The method comprises the steps of storing in a memory of a server (SER) a first set of data (F1) related to measurements performed on the wearer, in correspondence to a first identifier (ID1), and storing in the memory of said server (SER) a second set of data (F2) related to measurements performed on the frame to provide, in correspondence to a second identifier (ID2). The server (SER), upon reception of a piece of information (DOC2) indicating an association between the first and second identifiers, is arranged for compiling a computer document (ORD(ID1-ID2)) including at least a part of the first and second sets of data for enabling the edition of an order form.

The present invention relates to a method and a computer system and its components for providing a spectacle frame adapted to a future wearer.

Usually, a person needing to wear spectacles and having thus a prescription filled by an ophthalmologist or by another authorized eye care professional goes to the premise of an optician for choosing the frame of the future spectacles. The future wearer of the spectacles may try several spectacle frames and finally chooses one of the tried frames.

Usually, the optician performs measurements on the wearer while he is trying the chosen spectacle frames. These measurements are required to finalize the lens order when the lenses are to be delivered edged.

Usually, the inner circumference of the openings of the chosen frame (e.g. the openings of the frame where spectacle lenses are intended to be mounted) is measured very precisely by a mechanical sensor. More particularly, the openings of the frame include an inner rim and the characteristics of the rim (3D-shape of the rim, tilt angle with the openings, depth of the rim, etc.) are measured by the aforesaid mechanical sensor in a measuring room. The measurements performed by the mechanical sensor on the chosen frame make it possible to order spectacle lenses which fit the best the chosen frame, on the one hand, and the wearer prescription, on the other hand. More particularly, according to the measurements performed by the mechanical sensor and the optician in the measuring room, the optician and/or the provider of spectacle lenses are able to:
specify the edging job (shape, finishing options, etc.);
cut the edges of the lenses for fitting with the mechanical measurements performed on the chosen frame; and
provide spectacle lenses adapted to the wearer and the chosen frame.

The lenses provider has to ensure that the provided lenses would be adapted to the wearer and to the chosen frame. For example, the lenses provider has to ensure that the future lenses can effectively be mounted on the chosen frame which may have particular openings and rims.

It will be thus understood that the measurements performed on the inner circumference openings of the chosen frame is an important set of features to take into account for the lenses provider.

Therefore, usually, the mechanical measurements are performed on the frame that has been chosen by the wearer and exactly this frame. In fact, slight differences of mechanical features can be measured on two different frames of a same model and a same size. However, the lenses choice and their edging have to be very accurate and are very sensitive to those slight differences. It is thus highly preferable to carry out mechanical measurements on the frame which will be provided to the wearer with the lenses.

However, the exact frame which is intended to be provided to the wearer may not be available in the optician premise, at the moment when the wearer has chosen the frame. For example, the wearer may choose and try the model and the size of the frame but the color that the wearer prefers may not be available. The optician can measure centering parameters with an available frame tried by the wearer and write down manually the choice of the wearer and the measures performed when the wearer tried the available frame in the optician premise. In a second time, when the final frame is available (chosen model, chosen size, chosen color), the optician can measure the final frame and fill in the lens order. However, such an implementation is a source of delays (waiting for the final frame to arrive in the optician premise) and mistakes (linked to the manual process) and, thus provides a bad service to the wearer.

Moreover, the optician premise and the measuring room (and the spectacle frame inventory) may be located at different distant places. The optician may send to the measuring room the features of the frame chosen and tried by the wearer and all the order data in order to perform the measurements on the final frame of the exact model chosen by the wearer and to fill in the complete order. However, this implementation is again a source of mistakes and inconvenience as order data shall be transmitted manually between the optician premise and the measuring room.

The present invention aims to improve the situation.

To this end, it proposes a method performed by computer means and more particularly wherein a server centralizes the storage of the features of the frame chosen by the wearer, on the one hand, and the measurements performed on the frame intended to be provided to the wearer, on the other hand.

Therefore, the method according to the invention comprises the steps of:
storing in a memory of the server a first set of data related to measurements performed on the wearer, in correspondence to a first identifier,
storing in the memory of the server a second set of data related to measurements performed on the spectacle frame to provide, in correspondence to a second identifier.

More particularly, the server, upon reception of a piece of information indicating an association between the first and second identifiers, is arranged for compiling a computer document including at least a part of the first and second sets of data.

As the different steps for ordering the spectacles are centralized and controlled by the server, through the use of the first and second identifiers for managing the measurements files storage, the invention makes it possible to prevent from the aforesaid sources of mistakes. The optician may simply let choose the wearer his future spectacle frame, for example in a catalogue, and order consequently the chosen frame at a remote place where measurements are performed on a frame of a same model (and having the chosen size and color), and which is intended to be provided finally to the wearer.

It is thus to be understood that the frame chosen by the wearer and the frame on which the measurements are performed are not necessarily the same but may be, at least, of a same model (and, in some cases, of the same size).

Therefore, the two steps of the method according to the invention aiming respectively storing the first set of data and storing the second set of data can be performed asynchronously and in any order in time.

In an advantageous embodiment, the first set of data is sent to the server along with the aforesaid first identifier, but also along with a frame model chosen by the wearer. For example, the identifier of the model chosen by the wearer may be a data of the aforesaid first set of data.

In an advantageous embodiment, once the measurements are performed on the frame to be provided, the second set of data is sent to the server along with the first and second identifiers, providing thus the aforesaid piece of information indicating an association between the first and second identifiers.

In this advantageous embodiment, the server is thus informed of the pairing of the wearer information (including the chosen frame model) and the frame information (including measurements performed on the frame to provide to the wearer).

Other features and advantages of the invention will become apparent on reading the following detailed specification and examining the appended drawings, in which:

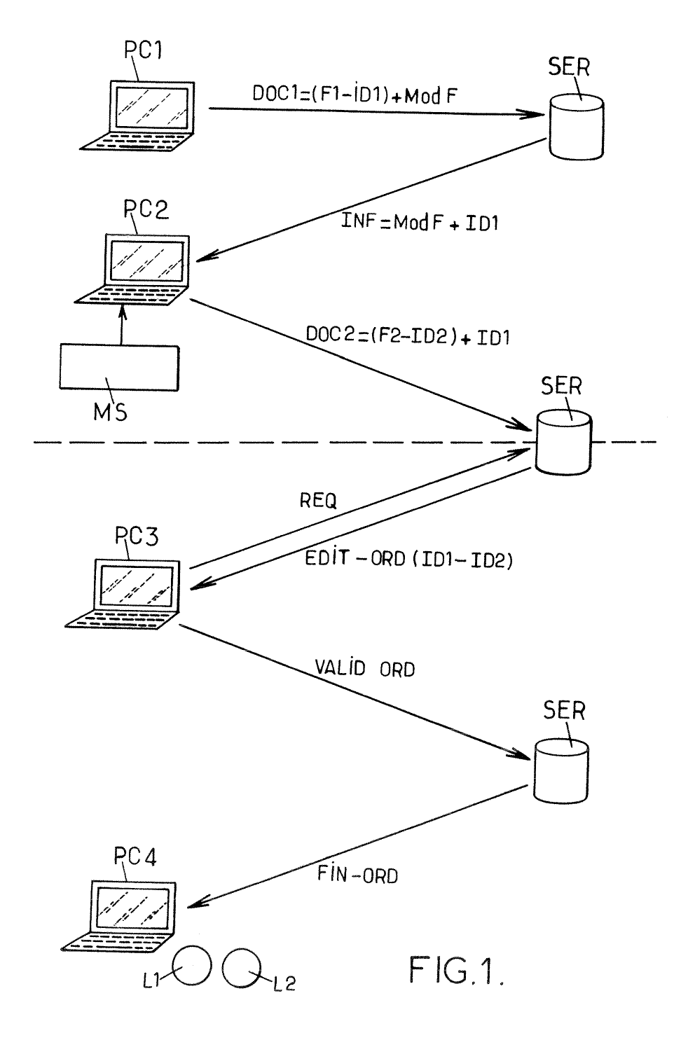

FIG. 1 is a diagram showing the interactions between the computer entities and the server involved in a method according to the invention;

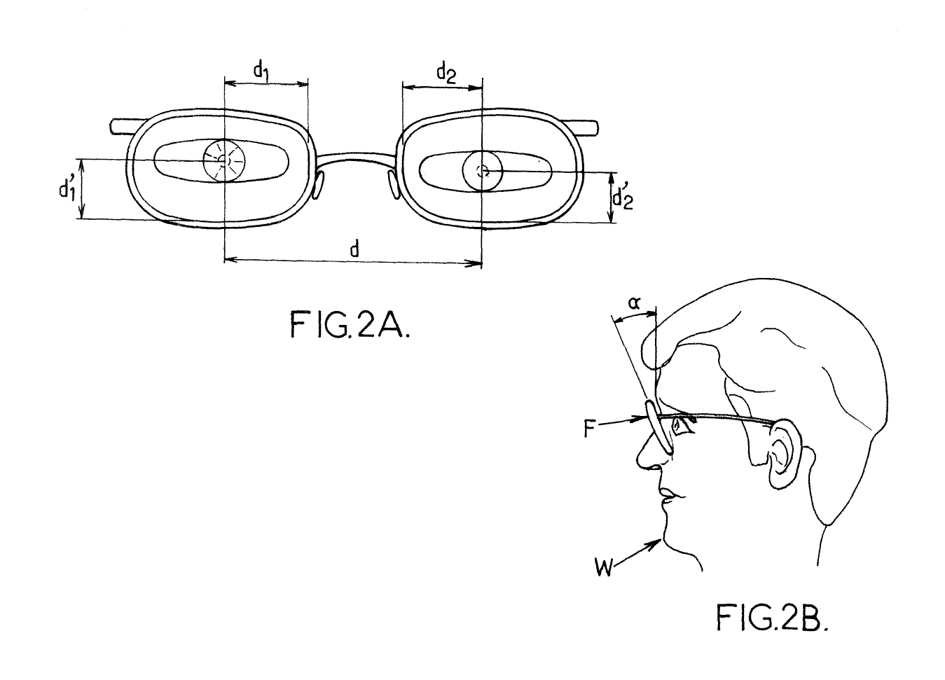

FIG. 2A and FIG. 2B are respectively front elevation and side elevation of the chosen frame F, showing in particular parameters to be measured for constituting the aforesaid first set of data;

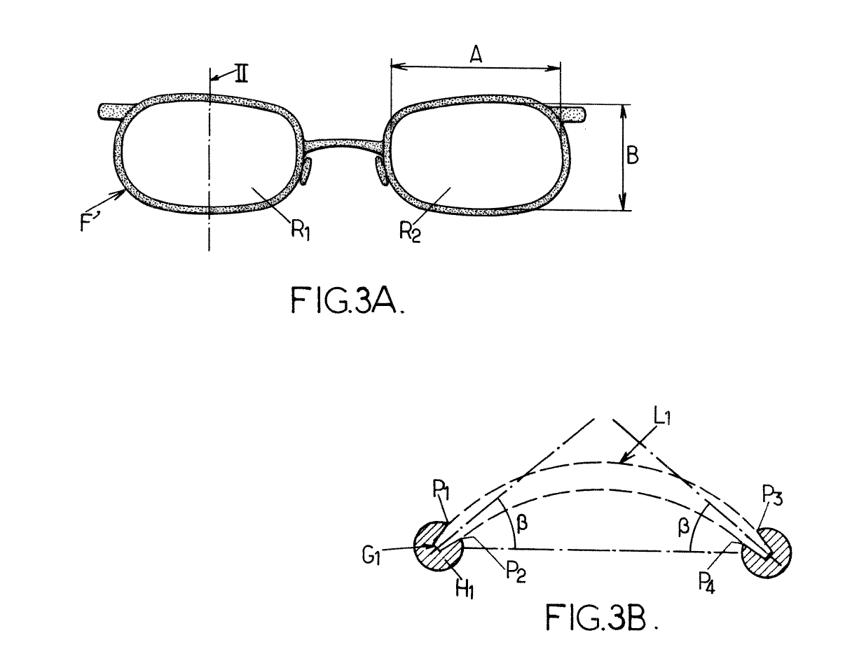

FIG. 3A and FIG. 3B are respectively a front elevation and a cross-section view of the frame to provide F′ (according to the trim mark II of FIG. 3A), showing in particular parameters to measure for constituting the aforesaid second set of data; and

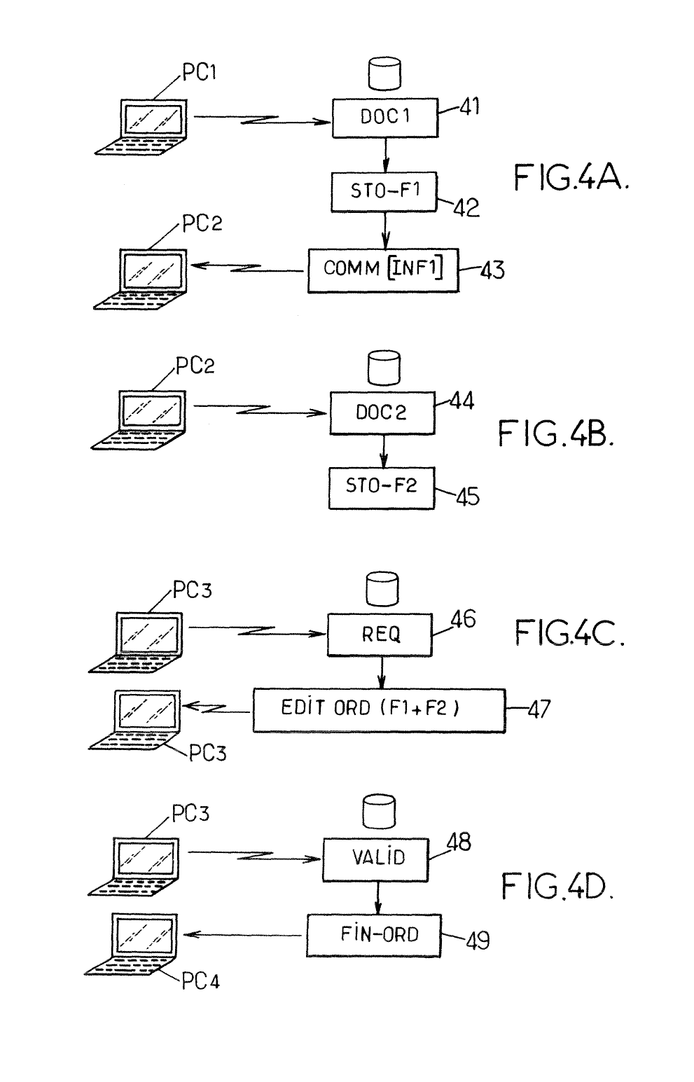

FIGS. 4A-4D show steps of the method performed by a server according to the invention, in an advantageous embodiment.

It is described hereafter a particular embodiment where the measurements performed on the wearer w (FIG. 2B) are carried out in a first location provided with a first computer entity PC1, while the measurements performed on the spectacle frame F′ (FIGS. 3A-3B) to provide are carried out in a second location, provided with a second computer entity PC2. With reference to FIG. 1, the first computer entity PC1, the second computer entity PC2 and the server SER are linked together via an extended network such as the Internet.

The measurements performed on the wearer w are stored in a computer file F1 constituting thus the aforesaid first set of data. To this end, the first computer entity PC1 comprises a memory for saving this first computer file F1. The computer entity PC1 includes also processing means for assigning to this first computer file F1 a name corresponding to the aforesaid first identifier ID1. Therefore, the first computer entity PC1 sends this first computer file F1 to the server SER along with the first identifier ID1 (for example as a name of the file F1). The first computer entity PC1 sends also, along with the first file F1, an identifier Mod F of a model of a frame tried and chosen by the wearer.

Therefore, the entity PC1 sends to the server a document DOC1 including the file F1 comprising the first set of data and the identifier Mod F of the model chosen by the wearer (step 41 of FIG. 4A). In step 42 of FIG. 4A, the server stores in a memory (non-volatile memory) the file F1 and the identifier Mod F. Then, the server, at step 43, sends to the second entity PC2 information combining the first identifier ID 1 to the identifier Mod F of the chosen frame. In another embodiment, the second computer entity PC2 may require from the server a list of waiting orders and an operator of the second computer entity PC2 may select one order in this list, for example for performing measurements on a frame to provide which is available in a premise where the second entity PC2 is located.

Thus, the second entity PC2 recovers from the server SER the first identifier ID1 along with the identifier Mod F of the chosen frame in step 43 of FIG. 4A. On the basis of the identifier Mod F of the chosen frame, an operator of the second computer entity PC2 uses a mechanical sensor MS to perform measurements on a frame F′ to provide to the wearer w, this frame F′ (FIGS. 3A-3B) to be provided to the wearer being at least of a same model as the frame F (FIGS. 2A-2B) tried and chosen by the wearer w. Once the measurements are performed on the frame F′ to provide, the measured values are stored in a second computer file F2. To this end, the second entity PC2 comprises a memory for saving this second computer file F2 comprising the aforesaid second set of data. The second computer entity PC2 comprises also processing means for assigning to the second computer file F2 a name corresponding for example to the aforesaid second identifier ID2.

Then, the second computer entity PC2 sends to the server SER the second set of data F2 along with the first identifier ID1 and the second identifier ID2 in the document DOC2 of FIG. 1 (step 44 of FIG. 4B). The server stores the second computer file F2 in step 45 of FIG. 4B. More particularly, the server, upon reception of this second document DOC2 is able to associate the first computer file F1 having a name corresponding to the first identifier ID1 and the second computer file F2 having a name corresponding to the second identifier ID2, making it possible to build an order form including data of the first set F1 and data of the second set F2.

In a particular embodiment, documents DOC1 and DOC2 can be in the form of html pages comprising tags corresponding to the aforesaid identifiers ID1, ID2, Mod F, and pointing at computer files F1, F2.

The aforesaid first set of data F1 may include data relative to the wearer (for example a pupillary distance d of the wearer (FIG. 2A), the prescription filled by an ophthalmologist, a coefficient of motion of an eye relatively to a global motion of the head, etc.) and/or data relative to a combination of the wearer w and the chosen frame F (for example heights d′1 and d′2 (FIG. 2A) between each wearer pupil and a horizontal edge of the openings of the chosen frame (called “fitting point height h” according to the specification ISO 13666), distances d1 and d2 between each wearer pupil and a vertical edge of the openings of the chosen frame (FIG. 2A), a pantoscopic angle α (FIG. 2B) between the vertical plan and the chosen frame F when the wearer w tries the spectacle frames, etc.).

The first set of data F1 may include also, for example, the width A and the height B of each rim of the frame F′, as shown on FIG. 3A. However, it is preferred to carry out these measurements directly on the frame F′ to provide and the values of these measurements may rather be included in the second set of data F2.

It is to be noted that the pupillary distance d measured on the wearer w makes it possible to position the optical centers in the frame in order to determine exactly the lens centering. The measurement of the coefficient of motion of an eye relatively to a global motion of the head makes it possible to determine lens design in order to adapt the look of the wearer according to his propensity to move the head or the eyes when looking in another direction.

Finally, the measurements stored in file F1 makes it possible to determine the centering of the lenses to provide with the spectacle frame of a same model and size of the chosen and tried one.

The aforesaid second set of data F2 is related to the frame F′ which is intended to be provided finally to the wearer. It may include in particular the values of the inner circumference measurements performed on the frame to provide by a mechanical sensor MS.

In an embodiment described here as an example, each spectacle lens L1 is intended to be mounted on a rim H1 delimiting an opening R1 of the frame F′ to provide (FIG. 3B). Thus, the aforesaid second set of data F2 may include for example an inner circumference measurement of the respective rims of the first and second openings R1, R2. More particularly, the second set of data may include for example:
dimension measurements of an internal groove G1 formed in the inner circumference of a rim H1 (FIG. 3B),
a measurement of an angle between a principal axis of the groove G1 and a principal axis of the rim H1 (i.e. a tilt angle β of the groove G1 relatively to the opening R1 intended to receive lens L1 in FIG. 3B).

The tilt angle β can be determined from the coordinate measurements of four points P1, P2, P3 and P4 as shown in the example of FIG. 3B.

The second set of data F2 may include also the width A and the height B of the rims of the frame to provide F′, if these data are not included already in the first set of data F1 as explained above.

Of course, the type of the chosen frame (for example a fully rimmed frame, or a semi-rimmed frame or a rimless frame) may be also a data to be included in the first set F1 or in the second set F2.

Referring again to FIG. 1, the server SER, upon a request REQ received from a distant computer entity PC3, is able to send to this computer entity PC3 data enabling an edition (“EDIT-ORD(ID1-1D2)”) by the computer entity PC3 of a computer document including the data of the first and second sets. This computer entity PC3 can be located in an optician premise that may be different from the previous two locations and is able to generate, upon reception from the server SER of the data enabling the aforesaid edition, an order form including both:
data related to measurements performed on the wearer w, on the one hand, and
data related to measurements performed on the spectacle frame to provide F′, on the other hand.

This computer entity PC3 can thus consult, online, the content of a list of waiting orders and, more particularly, can complete and validate an order form including the first and second data sets. In an advantageous embodiment, the server SER comprises processing means to calculate an optimum shape of the lenses to provide, according to the wearer measurements (including the prescriptions of the wearer) and the measurements performed on the frame to provide. Therefore, the order form may include characteristics of spectacle lenses intended to be mounted on the frame F′ to provide, as well as characteristics for edging these spectacle lenses, these lenses fitting both the features of the wearer and the features of the frame F.

It is to be noted that the computer entity PC3 can be the same as the first computer entity PC1. Therefore, the optician may run his computer entity PC1 (or PC3) for requesting the edition of an order form (step 46 of FIG. 4C). At step 47 of FIG. 4C, the optician can read on his computer screen (PC1 or PC3) the order form characteristics presented by the server. If the optician validates the order form (step 48 of the FIG. 4D), the server SER transmits the order form to a lens provider computer entity PC4 for ordering the pair of lenses L1 and L2 fitting both the wearer features and the features of the frame F′ to provide (step 49 of FIG. 4D).

According to an advantage provided by the invention, all the asynchronous steps of the spectacle ordering (frame ordered by the wearer, measurements on the frame to provide, choice of the lenses to provide) are organized and centralized by the server. It is to be noted also that the server is able to store all the measurements and data (computer files F1 and F2) in the whole ordering process. Thus, it will be understood that the storage of computer files F1 and F2, respectively by computer entities PC1 and PC2, can be volatile (for example a storage in a volatile memory).

The present invention aims also at a server comprising thus:
a memory for storing the first and second sets of data, and
processing means for compiling, upon request REQ, a computer document ORD (ID1-1D2) including data of the first and second sets, as described above with reference to FIG. 4C.

The present invention aims also at a software product intended to be stored in a memory of a processor unit of a server SER according to the invention, or in a removable memory medium adapted to cooperate with a reader of the processor unit of the server. In particular, the software comprises instructions for implementing the invention as described above with reference to FIGS. 4A-4D.

The present invention aims also at a computer entity amongst computer entities PC1, PC2, PC3 of FIG. 1. It aims at the entity PC2 for its ability to recover from the distant server the first identifier ID1 along with the identifier of the chosen frame Mod F, and for sending to the server, along with the first identifier ID1, the computer file F2 having a name corresponding to the second identifier ID2. The present invention aims also a software product adapted to be stored in a memory of a processor unit of a computer entity PC1, PC2 or PC3, or in a removable memory medium adapted to cooperate with a reader of the processor unit of the computer entity. This software product comprises instructions for implementing the steps of the method described above with reference to FIG. 1.

The invention aims also at a system for implementing the steps of FIG. 1, and comprising thus a server and at least one computer entity PC1, PC2 and/or PC3.

Of course, the present invention is not limited to the embodiment described herein above by way of example; it extends to other variants.

It has been described hereinafter documents DOC1 and DOC2 having the form of html pages. As a variant, a directory having a name corresponding to an identifier of the spectacles order may include two files F1 and F2 respectively corresponding to the first and second sets of data and having names corresponding respectively to the first and second identifiers. Such a directory can be transmitted for example from entity PC2 to the server, instead of an html page DOC2.

It is to be noted that computer entities PC1 and PC3 can be a same computer entity in the optician premise as explained above. Furthermore, computer entities PC1 and PC2 can be a same entity, as well, in particular if the optician is provided with a mechanical sensor MS for carrying out the measurements on the frame to provide. It will be thus appreciated that the data storage can be entrusted to a distant server, according to the invention.

It will be understood also that the chosen frame F and the frame to provide F′ can be a same and unique frame. The implementation of the invention can be advantageous again simply for entrusting the data storage to a distant server.

Moreover, the contents of the first and said sets of data are given herein above by way of examples and allow variants.

1. A method implemented by computer means, for providing a spectacle frame adapted to a future wearer of said spectacle frame, wherein the method comprises the steps of:
storing in a memory of a server a first set of data related to measurements performed on said wearer, in correspondence to a first identifier; and
storing in the memory of said server a second set of data related to measurements performed on said spectacle frame, in correspondence to a second identifier,
wherein the server, upon reception of a piece of information indicating an association between said first and second identifiers, is arranged for compiling a computer document including at least a part of said first and second sets of data.

2. The method according to claim 1, wherein said server is arranged, upon a request received from a distant computer entity, for sending to said distant computer entity data enabling an edition by said distant computer entity of said computer document.

3. The method according to claim 2, wherein said computer entity is located in an optician premises and is able to generate, upon reception from the server of said data enabling an edition of said computer document, an order form including both:
data related to measurements performed on the wearer, on the one hand, and
data related to measurements performed on the spectacle frame, on the other hand.

4. The method according to claim 3, wherein the data included in said order form define spectacle lenses intended to be mounted on said frame to provide, as well as characteristics for edging said spectacle lenses.

5. The method according to claim 4, wherein said order form, upon allowance by the optician, is transmitted by said server to a lens provider computer entity.

6. The method according to claim 1, wherein said spectacle frame on which measurements are performed is at least of a same model as a spectacle frame tried and chosen by said wearer.

7. The method according to claim 6, wherein said first set of data includes data relative to a combination of the wearer and said chosen frame and comprises at least one element among:
a height between a pupil of the wearer and a horizontal edge of said chosen frame,
a distance between a pupil of the wearer and a vertical edge of said chosen frame, and
a pantoscopic angle between the face of the wearer and said chosen frame.

8. The method according to claim 7, wherein said first set of data further includes data relative only to the wearer and comprises at least one element among:
a pupillary distance of the wearer,
a prescription of the wearer filled by an ophthalmologist, and
a coefficient of motion of an eye relatively to a global motion of the head.

9. The method according to claim 6, wherein said first set of data is sent to the server along with said first identifier and an identifier of a model of said chosen frame.

10. The method according to claim 1, first and second spectacle lenses being intended to be mounted on respective first and second rims of the spectacle frame to provide, wherein said second set of data comprises at least one element among:
an inner circumference measurement of said first and second rims,
a dimension measurement of an internal groove formed in said inner circumference of the rims, and
a measurement of an angle between a principal axis of said groove and a principal axis of a rim.

11. The method according to claim 10, wherein said second set of data is sent to said server along with said first and second identifiers, providing thus said piece of information indicating an association between said first and second identifiers.

12. The method according to claim 1, wherein said measurements performed on said wearer and said measurements performed on said spectacle frame are carried out respectively at two distinct and remote locations, respectively provided with a first computer entity and a second computer entity, said first and second computer entities being linked with said server via an extended network.

13. The method according to claim 12, wherein:
the first computer entity sends the first set of data to the server along with said first identifier and an identifier of a model of a frame tried and chosen by the wearer,
the second computer entity recovers from the server said first identifier along with the identifier of said chosen frame,
on the basis of said identifier of the chosen frame, the second computer entity orders measurements on a frame to be provided to the wearer, said frame to be provided being at least of a same model as the frame tried and chosen by the wearer, and
the second computer entity sends said second set of data to the server along with said first and second identifiers, providing thus to the server said piece of information indicating an association between said first and second identifiers.

14. A server for implementing the method according to claim 1, comprising:
a memory for storing:
a first set of data related to measurements performed on said wearer, in correspondence to a first identifier, and
a second set of data related to measurements performed on said spectacle frame, in correspondence to a second identifier; and
processing means being adapted for, upon reception of a piece of information indicating an association between said first and second identifiers, compiling a computer document including at least said first and second sets of data.

15. A software product adapted to be stored in a memory of a processor unit of a server, or in a removable memory medium adapted to cooperate with a reader of the processor unit of the server, comprising instructions for implementing the method according to claim 1.

16. A computer entity comprising means for implementing the method according to claim 2.

17. (canceled)

18. The computer entity according to claim 16, comprising:
a memory for saving a set of data in a computer file,
processing means for assigning to said computer file a name corresponding to a predetermined identifier, and for transmitting said file to a distant server,
and wherein said memory for saving said set of data in a computer file is a volatile memory.

19. A software product adapted to be stored in a memory of a processor unit of a computer entity, or in a removable memory medium adapted to cooperate with a reader of the processor unit of the computer entity,
comprising instructions for implementing the method according to claim 2.

20. (canceled)
